SEQUENCE LISTING (2 sequences)

SEQ ID NO: 1 - DNA, 19 bp, Artificial Sequence
  primer_bind: nucleotide sequence of upstream primer LMV355 promoter
aggacacgtgaaatcacca

SEQ ID NO: 2 - DNA, 34 bp, Artificial Sequence
  primer_bind: nucleotide sequence of downstream primer LDH-3′ n = any at positions 1, 2, 3, 4, 5
nnnnnggatcctactataactgcacatccttctg

(19) United States
(12) Patent Application Publication
(10) Pub. No.: US 2004/0088765 A1
(43) Pub. Date: May 6, 2004

(54) Animal immunocontraceptives expressed in plants and uses thereof

(76) Inventors: G. Barrie Kitto, Austin, TX (US); Daniel C. Hirschhora, Bosques de las Lomas (MX)

Correspondence Address: Benjamin Aaron Adler, ADLER & ASSOCIATES, 8011 Candle Lane, Houston, TX, 77071, US

(21) Appl. No.: 10/664,118

(22) Filed: Sep. 17, 2003

Related U.S. Application Data
(60) Provisional application No. 60/412,043, filed on Sep. 19, 2002.

(51) Int. Cl. A01H 1/00; C12N 15/82

(52) U.S. Cl. 800/288

(57) ABSTRACT

The present invention provides immunocontraceptive vaccines comprising a genetically engineered plant that has been modified to produce the sperm-specific protein lactate dehydrogenase-C (LDH-C). When animals such as rodents eat this plant, their immune systems produce antibodies that attack their sperms. Not only would the males have less viable sperm, the females would also have antibodies to sperm entering their reproductive systems. Immunocontraception is an attractive method for reducing the population size of animals with high fecundity, and sterilizing animals using such immunocontraceptives can reduce targeted animal populations to acceptable levels in an efficient, cost-effective, humane and, importantly, a species-specific manner.

### CROSS-REFERENCE TO RELATED APPLICATION

This non-provisional application claims benefit of provisional U.S. Ser. No. 60/412,043, filed Sep. 19, 2002, now abandoned.

### BACKGROUND OF THE INVENTION

1. Field of the Invention

The present invention relates generally to the field of immunocontraceptives. More specifically, the present invention relates to methods of controlling animal populations using immunocontraceptives expressed in plants.

2. Description of the Related Art

In the early 1990s, thirty billion dollars per year was lost due to food and property damage caused by rat and mouse worldwide. In the United States alone, rats and mice destroy almost $2 billion worth of foodstuffs and property annually. Insurance companies and government agencies spend hundreds of millions of dollars annually due to health complications and property loss caused by exposure to the pest.

Effort to control rodents is an ongoing and largely unsuccessful battle. The house mouse is capable of reproducing at 45 days old, though the mouse may not start breeding until up to ten weeks old. With a gestation period of 21 days and a normal litter size of five or six, a single pair of mice can produce a large number of offspring in a two-year lifespan. Mathematically, if that one pair reproduced every 24 days (a mouse needs three days after birth before she can re-mate), averaged 5.5 babies per litter, and each offspring began reproducing at 45 days old under the same circumstances, there would be 500 mice in only 21 weeks. In addition, the breeding season for mice is almost twice that long. Thus, mice breeding can suddenly overwhelm an area.

In contrast to mice, rats reach sexual maturity after 2-3 months, have a gestation period of 23 days, and average 20 young per year. Nevertheless, with either species, populations can increase dramatically given the availability of food, water, and a lack of natural enemies that can reduce their numbers.

When a consumer, farmer, or city official purchases a rodent control product, they would effectively like to buy a healthier and cleaner environment, as well as a decrease in the wear and destruction of their property. More specifically, consumers want rodent control products that eliminate the rodent population but do not produce harmful side effects to non-targeted animals or people. The preferred rodent control products should also not poison the environment and should be easy to use and maintain.

The two current methods of rat and mouse control—poisons and traps—do not meet the purchase criteria of a majority of customers. Poisons currently on the market will kill rodents in the short-term. However, as researchers in Australia discovered, by almost eliminating the rat population, the animals that naturally controlled the rat population were also starved. With the decline in the number of predators, the few remaining rats reproduced much more quickly than the remaining predators. In fact, after the administration of poison, rats reproduced at faster rates than they did under non-poison conditions, resulting in a new massive wave of rat infestations. Poisons are also not species-specific, often harming other non-targeted animals and people. Furthermore, poisons often contaminate food stores and groundwater, further creating health risks to a broader population.

Alternatively, traps do not poison the environment. Traps are not, however, as effective as poisons in immediately reducing the rat population, and they are much more difficult to administer than poison bait systems. Pest control professionals must set traps, monitor them, remove the trapped rodents, and reset the traps.

For some time now, researchers have been trying to develop a method to decrease rodent fertility as a means of rodent control. Modeling has shown that if ⅔ of females are sterilized, mouse populations would double every twelve weeks instead of eight. That means at 35 weeks, the size of this mouse population would be half of an untreated mouse population. A potential approach for rodent sterilization is by inducing immune responses against the reproductive system of the rodent.

In the general area of immunocontraceptives, zona pellucida 3 (ZP3) has received much attention. Zona pellucida 3 is an egg-specific protein, so immunization with zona pellucida 3 would induce the immune system to attack the animals' own eggs. The uses of zona pellucida, however, has several drawbacks. First, zona pellucida 3 is not species-specific. Furthermore, as a method of distribution, researchers have tried to use murine cytomegalovirus (MCMV), a virus that only exists in mice and rats, to deliver zona pellucida 3 to the rodents. By genetically altering the virus, the zona pellucida-immunocontraceptive can theoretically be delivered directly to the rodents. The problem with this method, however, is that once the virus is released, it is irretrievable. Thus, concerns have been expressed over whether the virus could evolve and lose species-specificity over time, as well as whether the virus would result in complete extinction of the targeted rodents. In addition, failure to gain public acceptance and legal issues will pose major hurdles in introducing this technology (Seamark, 2001).

Hence, the prior art is deficient in methods of controlling animal populations using immunocontraceptives expressed in plants. The present invention fulfills this long-standing need and desire in the art.

### SUMMARY OF THE INVENTION

The present invention provides a fertility control agent that is cost-effective, humane and species specific for the control of animal populations. The fertility control agent comprises a genetically engineered plant that has been modified to produce the sperm-specific protein lactate dehydrogenase-C (LDH-C). When animals such as rodents eat this modified plant, their immune system produces antibodies that attack their sperms. Because the antibodies react to sperm, the instant fertility control agent affects the fertility of both males and females. Not only would the males have less viable sperms, the females would also have antibodies to the sperms entering their reproductive systems. The induced sterility is only maintained as long as the animals ingest the bait. Therefore, there is no concern over extinction of the targeted animals or of their predators.

In one embodiment of the present invention, there is provided a genetically modified plant that expresses an immunocontraceptive comprising an egg- or sperm-specific polypeptide. The contraceptive agent is useful in controlling the size of an animal population by inducing sterility in animals that have ingested the genetically-engineered plant materials.

In another embodiment of the present invention, there are provided methods of using these contraceptive agents to control the population size of animals that has reached pest levels.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows a modeling of rodent population size after treatment with 60% or 80% effective contraceptive. The constraints used were: initial population=20 mice (10 female, 10 male); average lifespan=1 year; gestation time=20 days; fecundity, 5 litters/year, 8 offspring/litter; time to sexual maturity from birth=45 days.

FIG. 2 is a map of pLBJ21-LDH-C. The plasmid is a modified Agrobacterium plasmid capable of expressing lactate dehydrogenase-C (LDH-C) in plants.

FIG. 3 shows expression of lactate dehydrogenase-C in Arabidopsis thaliana: Extracts from plants of clones 1, 4, and 11 or wild type (wt) were prepared for western blots. The immunoblots were probed with a rabbit anti mouse lactate dehydrogenase-C. Mouse testes were used as the postitive control.

FIG. 4 shows native gel of extracts stained with a tetrazolium salt. Lane 1, mouse testis extract; lane 2, rLDH-C 10 μl; lane 3, rLDH-C 5 μl; lane 4, wild type tobacco extract; lane 5, transgenic plant #1 extracts expressing lactate dehydrogenase-C; lane 6, mouse testis extract; lane 7, transgenic plant #2 extract expressing lactate dehydrogenase-C; lane 8, trangenic plant #3 extract expressing lactate dehydrogenase-C. The transgenic plants expressed active lactate dehydrogenase-C by comparison to mouse testis extracts or to the wild type enzyme. The small band on the wild type lane was believed to be contamination from an overloaded rLDH-C adjacent lane. Recombinant lactate dehydrogenase-C migration was further than native lactate dehydrogenase-C since the recombinant protein was genetically fused to a highly positively charged histidine tag.

FIG. 5 shows serum IgG levels during the period of the vaccine trials. IgG levels were tested by indirect ELISA on the days shown. The groups reprsent the average response of groups of 10 or 5 mice After the last immunization a specific immune response can be seen in both 150 and 75 μg lactate dehydrogenase-C dose. A modest cross reaction response was seen in wild type (wt) tobacco extracts due to the large amount of antigenic protein that was contained in the extracts.

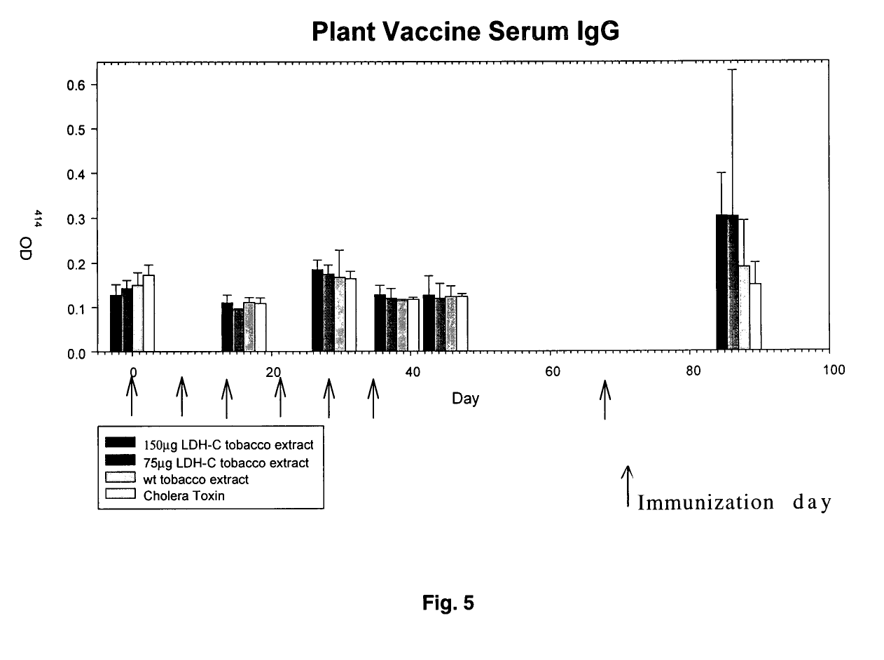

### DETAILED DESCRIPTION OF THE INVENTION

The present invention discloses the development of new methods for the control of animal populations that reach pest proportion. These methods of the present invention incorporate recently developed immunocontraception technologies. The basic premise of immunocontraceptives is to orally immunize animals by using bait formulations containing proteins from the animals' own reproductive system so that immune responses that block fertilization are induced in the animals after ingestion of such baits. Immunocontraception is an attractive method for reducing the population size of animals with high fecundity, and it is believed that sterilizing animals using such immunocontraceptives can reduce targeted animal populations to acceptable levels in an efficient, cost-effective, humane and, importantly, a species-specific manner.

Dr. Erwin Goldberg and his associates have been developing contraceptive vaccines for humans based on the development of immune responses to the sperm specific antigen lactate dehydrogenase-C (LDH-C). The rationale for this approach is based on the observation that circulating antibodies to sperm in both men and women are responsible for at least 10% of cases of idiopathic infertility (reviewed in Alexander and Bialy, 1994). Therefore, the use of a vaccine consisting of a defined antigen of sperm is likely to provoke immune reactions that lead to loss of fertility. In a series of studies over the past several years, Goldberg reported the anti-fertility effects of immunization with the sperm-specific isozyme of lactate dehydrogenase C4 (LDH-C4). In animal trials with female rabbits, baboons, mice and foxes up to 85% infertility has been obtained (O'Hern et al., 1995; Goldberg et al., 1981). While marginal for human use, this level of contraception would be satisfactory for control of pest populations of animals.

Experiments have been conducted to assess antibody response after different routes of immunization (i.e., systemic vs. local) (Alexander and Goldberg, 1992; Shelton and Goldberg, 1985). Generally, it was observed that IgG was produced both locally at mucosal surfaces and in the circulation. In fact, there are substantial reasons to believe that oral administration with concomitant development of mucosal antibody responses may be even more effective for contraceptive purposes than is the induction of a humoral IgG response. For example, Alexander and Bialy reviewed contraceptive vaccine developments and observed that oral administration can lead to immune responses in other areas of the mucosal immune system and that through oral administration it should be possible to induce high antibody titers in the reproductive tract.

Although the use of a contraceptive vaccine is a new approach to rodent control, the concept of bait borne vaccines is an established practice. For example, the Texas Department of Health routinely airdrops food pellets containing rabies vaccine to control this disease in gray foxes and coyotes.

The availability of complete amino acid sequence information and high-resolution crystal structure for the lactate dehydrogenase-C4 (LDH-C4) antigen has allowed for B-cell epitope mapping of the molecule and subsequent synthesis of species-specific immunodominant epitope peptides. Immunodominant epitopes of lactate dehydrogenase-C4 can be identified by two complementary methods. The first one consists of computer algorithms for B-cell epitope prediction (Van Regenmortel and Daney de Marcillac, 1998). The predicted epitopes can be corroborated by sequence comparison with related mammalian lactate dehydrogenase-C immunodominant epitopes. The human, baboon, mouse, and rabbit share the most immunodominant epitope 5-15 amino acids at the N-terminus of the molecule (O'Hern et al., 1995; O'Hern et al., 1997).

Therefore, full length as well as antigenic fragments of the lactate dehydrogenase-C4 (LDH-C4) antigen can be incorporated into the immunocontraceptive of the present invention. As used herein, “fragment,” as applied to a polypeptide, will ordinarily be at least 8 residues, more typically at least 40 residues in length, but less than the entire, intact sequence. Fragments of the lactate dehydrogenase-C4 protein can be generated by methods known to those skilled in the art, e.g., by enzymatic digestion of naturally occurring or recombinant lactate dehydrogenase-C4 protein, by recombinant DNA techniques using an expression vector that encodes a defined fragment of lactate dehydrogenase-C4, or by chemical synthesis.

Fragment of lactate dehydrogenase-C4 have been synthesized in combination with additional elements such as diptheria toxoid or a promiscuous T-cell epitope of tetanus toxin and shown to have comparable contraceptive effectiveness to lactate dehydrogenase-C4 in several species (O'Hern et al., 1995; O'Hern et al., 1997). Other carriers considered include the use of complete cholera toxin, its beta subunit, or a genetically or chemically detoxified form of the toxin. This agent has been shown to be the strongest mucosal adjuvant and can be more adequately used when oral and mucosal vaccines are considered (Fujihashi, 2002). In a similar fashion, mucosal adjuvants from bacterial toxins or other sources could be used (Piazza, 2001).

In one embodiment of the present invention, there are provided immunocontraceptives useful in controlling rodent populations. Using some simplifying assumptions in a modeling of contraceptive effects on mouse populations, some calculations were made of how vaccines of 60% and 80% effectiveness might affect the population growth of a small starting population of mice in a habitat without resource constraints. This is a circumstance which might exist when mice initially infest a granary.

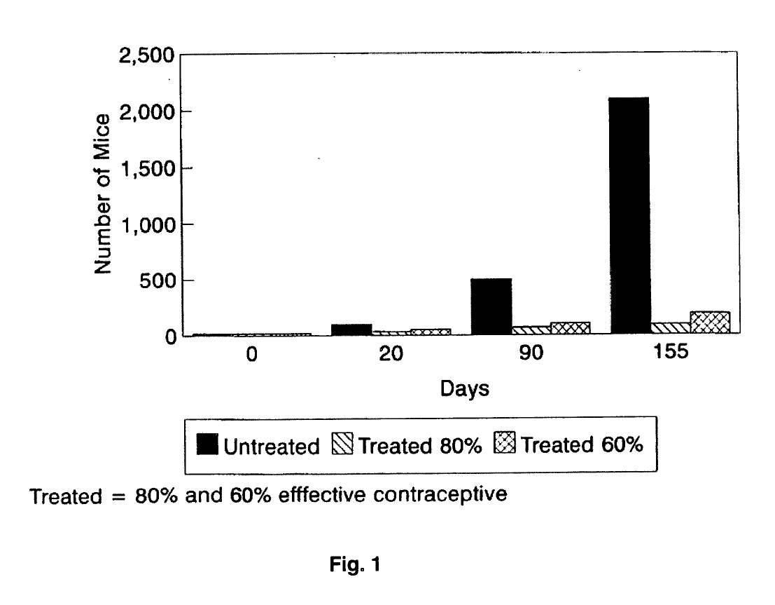

The results of such modeling are depicted schematically in FIG. 1, dramatically illustrating how an oral contraceptive of even 60% effectiveness can alter the rate of mouse population growth. The constraints used were: initial population=20 mice (10 female, 10 male); average lifespan=1 year; gestation time=20 days; fecundity, 5 litters/year, 8 offspring/litter; time to sexual maturity from birth=45 days.

One of ordinary skill in the art would recognize that the present invention is equally applicable to other animal or pest populations. Other animals to which this technology will apply include any mammalian species capable of eliciting an immune response against proteins of their own reproductive system. In particular deer, elephants, water buffalo, feral horses, foxes, urban or wild dogs, urban or wild cats, rabbits, and other potentially overpopulated species causing economic damage to society could be targeted.

Crucial to the development of a successful contraceptive bait is the selection of a proper carrier for the immunological agent. The carrier should be stable, capable of being inexpensively and efficiently produced, environmentally safe and attractive as a food to the targeted animals. Based on these requirements, the present invention uses plant materials as a vector for the immunocontraceptives.

Expressing antigens in plants that upon consumption can elicit a strong immune response, with effective protection against a variety of pathogens, has been well documented. Examples include the heat labile enterotoxin from E. coli, Cholera Toxin B subunit, Hepatitis B Surface Antigen, Norwalk Virus Capsid Protein (Manson et al., 1996; Haq et al., 1995; Arakawa et al., 1998). There are considerable advantages in the use of dietary immuncontraceptives for animal pest control. Not only can baits based on these plant materials be directly placed in feeders at strategic sites that pose no harm to the local environment, they also act as oral antigens capable of stimulating local mucosal immunity that augments and prolongs the immune effects (Manson et al. 1996, Haq et al. 1995, Arakawa et al. 1998).

In one embodiment of the present invention, there are provided successfully constructed Arabidopsis and tobacco strains expressing mouse lactate dehydrogenase-C up to 0.01% of total soluble protein. This recombinant protein showed comparable biochemical and immunological properties to its native counterpart and to the E. coli overexpressed protein. Extracts prepared from these plants were fed to mice and in preliminary studies most animals produced lactate dehydrogenase-C specific antibodies. Other plants that can be used include any plants that potentially can be made transgenic with the current and future technologies. In particular, these plants include potato, tomato, corn, banana, wheat, rice, any fruit, vegetable, legume, or grain crop.

In accordance with the present invention there may be employed conventional molecular biology, microbiology, and recombinant DNA techniques within the skill of the art. Such techniques are explained fully in the literature. See e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

As used herein, “immunocontraceptive” refers to an immunogenic composition comprising an antigen that can induce immune responses against the cells of an animal's reproductive system, thereby leading to loss of fertility in the treated animal.

The present invention is directed to a genetically modified plant that expresses an immunocontraceptive comprising an egg- or sperm-specific polypeptide or antigenic fragment thereof. The sperm-specific polypeptide includes lactate dehydrogenase-C such as rat or murine lactate dehydrogenase-C. These contraceptive agents are useful in controlling the size of an animal population by inducing sterility in animals that have ingested the genetically-engineered plant materials. In general, the plant materials include potato, tobacco, rice, bananas, wheat, corn, tomato, any fruit, vegetable, or legume. Representative polypeptides of sperm-specific lactate dehydrogenase-C include amino acids 5-17, 44-58, 61-77, 97-110, 180-210, 211-220, 231-243, 283-306, 307-316, 101-115 of murine lactate dehydrogenase-C (Hogrefe, 1987).

The present invention is also directed to methods of using these contraceptive agents to decrease the fertility of an animal. These contraceptives are first dispersed as baits in the habitats of the targeted animals, and ingestion of these baits by the animals would induce sterility in those animals. Susceptible animals include mice, rats, deer, elephants, water buffalo, feral horses, foxes, urban or wild dogs, urban or wild cats, rabbits, and other potentially overpopulated species causing economic damage to society.

The following examples are given for the purpose of illustrating various embodiments of the invention and are not meant to limit the present invention in any fashion.

### EXAMPLE 1

### Plasmid Cloning and Transformation into Agrobacterium tumefaciens

Transgenic plants were developed via Agrobacterium mediated transformation. The antigenic genes need to be cloned into a modified form of the Ti plasmid from Agrobacterium tumefaciens (Schardl et al., 1987b) to simplify the expression of foreign genes in plants. The plasmid was further engineered to contain EcoRI and HindIII sites and was called pLBJ21.

Plasmid pDNA3.1-LDH-C was provided by Dr. Erwin Goldberg from Northwestern University, Evanston, Ill. This plasmid contains the cDNA of mice lactate dehydrogenase-C between two EcoRI sites. The plasmid was transformed into XL-1 blue electrocompetent cells (Stratagene). The plasmid was isolated using the Quiaprep Spin Plasmid kit (Quiagen) following the manufacturer's instructions. The isolated plasmid was subjected to restriction digest with EcoRI and the LDH-C (lactate dehydrogenase-C) sequence was recovered by agarose gel purification. The lactate dehydrogenase-C sequence was then cloned into the plasmid pLBJ21.

To prepare the plasmid pLBJ21 for cloning, the plasmid was first obtained from Dr. Allan Lloyd (University of Texas at Austin). Plasmid pLBJ21 was provided transformed into DH5α cells (Novagen). A 10 ml LB culture containing 15 μl of tetracycline (5 mg/ml) was inoculated and grown with aeration at 37° C. overnight. The plasmid was isolated using the Quiaprep Spin Plasmid kit (Quiagen) following the manufacturer's instructions. Since pLBJ21 is a low copy plasmid, a 10 ml LB culture had to be used. The plasmid was subjected to restriction endonuclease digestion with EcoRI and was used as vector for cloning.

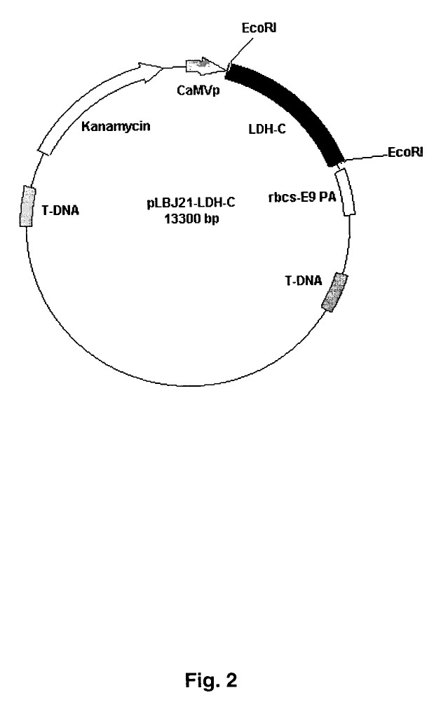

After a plate with ˜100 colonies was obtained from the cloning procedure, the cells were PCR screened for vectors containing the insert in the correct orientation. Since only one restriction enzyme was used, the gene could be inserted in the reverse direction. The colonies were PCR screened using the upstream primer LMV355Promoter (5′AGGACACGTGAAATCACCA) (SEQ ID No. 1) which is complementary to the Cauliflower Mosaic Virus Promoter and anneals to the vector. As a downstream primer the LDH-C3′ was used (5′NNNNNGGATCCTACTATA ACTGCACATCCTTCTG) (SEQ ID No. 2). The new plasmid was called pLBJ21-LDH-C (FIG. 2). The suspected positive colonies were used to inoculate a 10 ml LB culture with 15 μl of tetracycline (5 mg/ml) and the plasmid was isolated using the Qiaprep Spin Plasmid kit (Qiagen). The plasmids were submitted for sequencing to confirm the correct lactate dehydrogenase-C sequence was contained in pLBJ21-LDH-C.

After the correct sequence was confirmed, the plasmid was ready for transformation into the Agrobacterium. The strain GV3101(pMP90) of this bacterium (Knocz and Schell, 1986) was frozen in LB media supplemented with 10% glycerol. From this glycerol stock a 5 ml culture was inoculated and grown overnight in a 30° C. incubator shaking at 200 rpms. A dense culture was obtained next morning and was used to make a stock of electrocompetent Agrobacterium. One μl of each plasmid (approximately 50 ng) was mixed with 20 μl of freshly prepared cells and the mixture was incubated on ice for 30 minutes. The cells were transferred to an electroporation chamber Micro Electro Chamber (Gibco BRL Life Technologies). The chambers were placed in the Cell Porator Electroporation System I (Gibco BRL Life Technologies) and the transformation was performed according to the manufacturer's instructions.

The transformed Agrobacterium were recovered in 250 μl of SOC media at 30° C. while shaking at 200 rpm for 1 hour. The cells were further plated in double selection LB agar plates containing 50 mg/L kanamycin and gentamycin. The plates were incubated at 30° C. for 48 hours. About 10 colonies from each plate were PCR-screened. PCR was performed in a Perking Elmer Gene Amp PCR system 2400 using the following temperature sequence: 5 minutes, 94° C.; 30 cycles of 45 seconds, 94° C., 45 seconds, 55° C., 1.5 minutes, 72° C.; 10 minutes, 72° C. After PCR, positive clones were visualized on agarose gel. It is important to run a positive control (the original insert) and a negative control (water) so bands in the gel can be compared accordingly. Successful transformations were kept at −80° C. in glycerol stocks until further use.

### EXAMPLE 2

### Development of Transgenic Arabidopsis Thaliana by the Vacuum Infiltration Technique

Adult Arabidopsis were transfected with the modified Agrobacterium by applying a vacuum forcing transfection as described below. During transfection, the T-DNA plasmid was incorporated into the chromosome of the plant. The progeny of these plants will be transgenic and can be selected using an appropriate antibiotic.

On the first day of the experiment about 25 seeds from the laboratory wild type strains Wassilewskija (WSwt) and Landsberg erecta (Lerwt) of Arabidopsis thaliana were sprinkled in 12 small pots containing autoclaved soil that was previously moist with tap water. The planted seeds were further soaked with tap water and were covered with a plastic lid. The pots were covered from light and were placed at 4° C. for 2 days. This ensured all the plants would germinate uniformly. On the 3^{rd} day the pots were moved to a 20-22° C. incubator with a constant 24-hour exposure to light. The lid was not removed for 3-4 days or until the seeds were fully germinated. Small leaves and stems were visible at this time. The plants were kept in this incubator for the rest of the experiment and were watered and fertilized regularly. Approximately 4-5 weeks after planting, the adult plants were ready for transfection. The primary inflorescence should be 5-15 cm long and the secondary inflorescences should be appearing at the rosette.

A 125 ml LB culture containing 50 mg/L kanamycin and gentamycin was inoculated with previously prepared glycerol stocks of Agrobacterium and grown for 2 days at 30° C. with constant shaking at 200 rpm. The cultures were harvested by centrifugation at 500 g at 4° C. for 10 minutes and the supernatant was discarded. The bacteria were resuspended in 250 ml of infiltration medium composed of 2.2 g Murashige and Skoog salts (Sigma), 1×B5 vitamins (Sigma), 50 g sucrose (Sigma), 0.5 MES (Sigma) pH 5.7-5.8, 0.044 μM benzylaminopurine (Sigma), and 200 μl of the surfactant Silwet L-77 (Lehle Seeds) per liter (Bechtold and Pelletier, 1998). A 1000× stock of B5 vitamins consists of 1 g myoinositol, 0.1 g thiamine HCl, 10 mg nicotinic acid, and 10 ml pyridoxine HCl per liter.

The 250 ml infiltration medium with the Agrobacterium was transferred to a plastic 250 ml beaker in a vacuum dome. The plants were submerged in the medium upside down being careful not to introduce soil from the pots. The vacuum dome was sealed and a vacuum was created using an electric vacuum pump for 15 minutes. After infiltration, the plants were dripped to remove extra medium and kept in a moist environment for a day by replacing the plastic lid. The treated Arabidopsis were handled as previously described until the plants were seeding. At this point, watering was stopped and the seeds were collected from the dried plants. The seeds were dried in a dessicator for 3 days.

The seeds were ready for selection at this point and they were sterilized by placing them in a folded piece of filter paper closed accordingly to make a “tea bag”. The filter paper was submerged in a solution of 20% bleach with 0.1% tween 20 (Sigma) for 15 minutes. They were washed afterwards 3 times with distilled water. After the seeds were sterilized they were placed and distributed evenly in GM plates containing 100 mg/L of kanamycin (Sigma) and timetin (SmithKline Beecham) under sterile conditions. The GM plates were composed of 0.5× Murashige and Skoog salt mixture (Sigma), 1% sucrose, MES pH 5.7 (Sigma), and 0.8% tissue culture grade agar (Sigma). The plates were kept sealed with Parafilm at 4° C. in the dark for 2 days. On the third day, the plates were moved to an incubator at 22° C. with a 24-hour light cycle. The plates were incubated for 7-10 days until the seeds that were transformed germinated. The small plants were transferred to pots containing autoclaved, moist soil. Up to 30 plants were planted in each pot. The pots were covered with a transparent lid for an additional 3 days after which the transgenic plants were grown normally under the described conditions.

### EXAMPLE 3

### Development of Transgenic Tobacco

This method was adapted from the protocol developed by Horsch et al. (1985). It used a plant tissue culture format to transform explants. The strain of Nicotiana tabaccum Xanti N′N′ was used for transfection. The seeds were sterilized as described previously in the Arabiodpsis method. They were further plated on MS104 agar under sterile conditions and kept in an incubator at 22° C. with constant 24-hour light. MS104 plates consist of 2.2 g Murashige and Skoog's salts (Sigma), 3% sucrose, 0.5 g MES pH 5.7, 0.1 g/L naphthalene acetic acid (Sigma), 1.0 g/L benzyladenine (Sigma), and 0.8% tissue culture grade agar. The seeds germinated for about 2 weeks until the plants grew shoots that were approximately 1 cm long. At this point the plants were ready for transfection. The explants were prepared by excising the cotyledon from the plants leaving behind the hypocotyl and the outer portions of the leaf. The pieces of explants, excluding the cotyledon, were placed in fresh MS104 agar plates assuring that each piece of explant was in contact with the medium.

An Agrobacterium culture was prepared the day before transfection. For this, a 3 ml LB culture supplemented with 50 mg/L of kanamycin and gentamycin was inoculated with Agrobacterium from a frozen glycerol stock. The cultures were placed in a shaker incubator at 30° C. spinning at 200 rpm overnight. A turbid culture was recovered and 100 μl of it were added to 3 ml of liquid MS104 medium. The Agrobacterium mixture was added to the plates containing the explants ensuring that the liquid covered the explants in their entirety. The excess liquid was removed by aspiration, the plates were sealed, and they were placed in an incubator as described above. After 72 hours, the transfected explants were moved under sterile conditions to fresh MS104 agar plates containing 100 mg/L kanamycin and 100 mg/L timetin. Under these conditions the transformants grew into undifferentiated tissue forming tumor. After two weeks visible stems and leaves developed from each tumor. The plant tumors were moved to MS104 agar plates without hormones (naphthalene acetic acid and benzyladenine). This promoted the formation of roots. The plants were incubated for about a week until roots were formed. The transformants were then moved individually to pots containing moist soil under a humid atmosphere. This was accomplished by placing a transparent lid covering the pots. After 4-6 days, the plants started to adapt to their new soil environment and the lid was removed. The plants were further housed indefinitely at the University of Texas of Austin greenhouse facility where they were watered and fertilized regularly.

### EXAMPLE 4

### Expression of Lactate Dehydrogenase-C in Arabidopsis

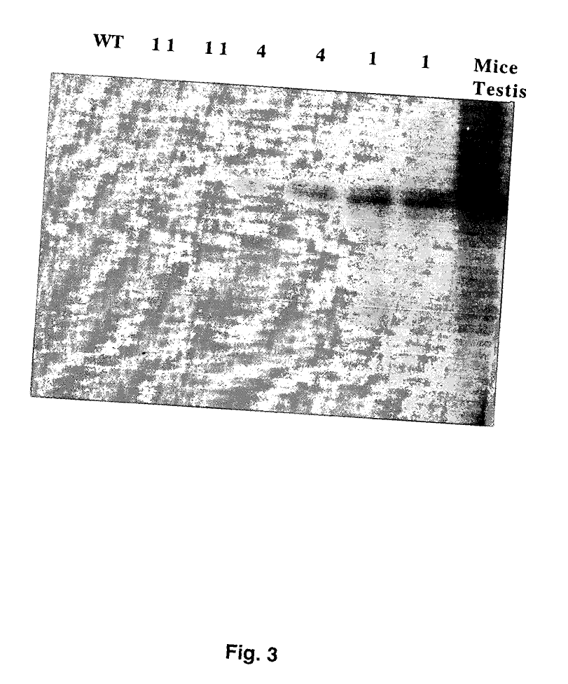

The expression of mouse lactate dehydrogenase-C (LDH-C) by transgenic Arabidopsis was tested using immunoblots. This required extraction of protein from the plants and polyacrylamide gel electrophoresis (PAGE). The protein preparation was performed using a protocol developed by Tanaka and Hurkman (1986). About 3 grams of plant material (leaves, stems, and roots) were frozen at −80° C. and ground with a mortar and pestle in the presence of liquid nitrogen until the plants were pulverized. At this point, 2 ml of extraction buffer (0.7 M sucrose, 0.5 M Tris, 30 mM HCl, 0.1 KCl, 2% β-mercaptoethanol, 10 mM phenylmethylsulfonyl fluoride (PMSF, Sigma), and 10× Complete Protease Inhibitor Cocktail (Roche)) were added and the grinding continued. The pulp was transferred to a Corex centrifuge tube and an additional 6 ml of extraction buffer was added. The tubes were incubated for 30 minutes at 4° C. and later centrifuged at 5,000 rpm for 10 minutes. The supernatant was transferred to a fresh Corex tube and was vigorously mixed with an equal volume of water-saturated, ice-cold phenol. The mixture was incubated for 45 minutes at 4° C. Phases were separated by centrifugation at 6,000 rpm for 10 minutes. The phenol phase and the interphase were transferred to a third Corex tube and 5 volumes of 0.1 M ammonium acetate in ice-cold methanol were added to it. Proteins were precipitated overnight at −20° C. and further isolated by centrifugation at 5,000 rpm for 10 minutes. The pellet was air-dried and was further resuspended in phosphate buffered saline (PBS) for further testing. Thirty μl of the extracts were diluted in 4×PAGE loading buffer, boiled for 5 minutes, and loaded on a 10% polyacrymalide gel. After the gel was finished, the proteins were transferred to a polyvinylidenefluoride (PVDF) membrane (Pierce). For Western Blot analysis, the primary antibody was an affinity purified rabbit anti-mouse lactate dehydrogenase-C (provided by Dr. Erwin Goldberg) used at 1:10,000 dilution. For the secondary antibody, a commercial grade goat anti-rabbit IgG-HRP (Kirkegaard and Perry Laboratories) was used at the recommended 1:10,000 dilution (FIG. 3).

Protein expression can also be examined by native gel staining with tetrazolium blue. Lactate dehydrogenases can be detected using a colorimetric assay in which the reduction of NAD is coupled to the reduction of a tetrazolium salt providing a red precipitate. This method can be performed in a polyacrylamide gel electrophoresis system using non-denaturing gels. This method is an adaptation of the original protocol developed by Arthur Babson and Susan Babson (1973). A 10% native polyacrylamide gel was prepared, and no SDS was used in any of the buffers nor an oxidizing agent like dithiothreitol was used. The protein samples were not boiled and precautions were taken to ensure the proteins were native and active.

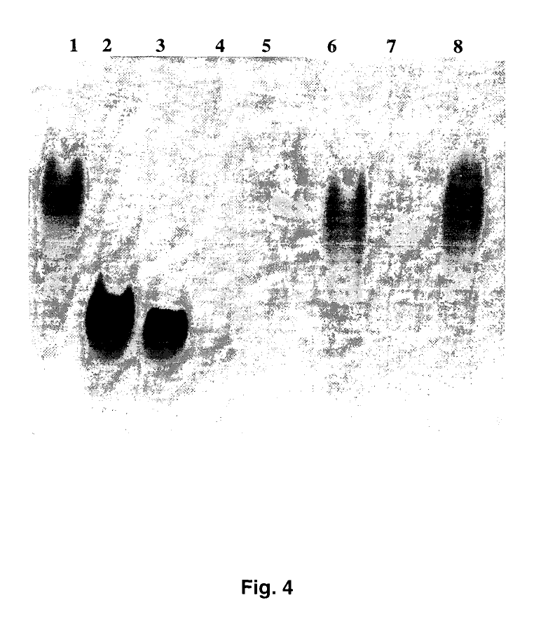

Protein extracts were prepared as those for lactate dehydrogenase-C expressing plants. The extracts were mixed with 4× native loading buffer and loaded in a discontinuous Tricine-sodium dodecyl sulfate-PAGE designed for optimal peptide separation by Schagger and von Jagow (1987). The samples were run at 125 volts with constant amperage for 3 hour. The gel was washed once with deionized water and then submerged into 20 ml of substrate solution consisted of 50 mM Tris-HCl pH 8.2 and 50 mM L(+) lactic acid solution (Miles Chemical Co., Clifton, N.J. The gel was incubated for a few minutes at 37° C. and then color reagent 40 mg of INT (2-p-iodophenyl-3-p nitrophenyl-5-phenyl tetrazolium chloride) (Sigma), 100 mg of NAD (Sigma) and 10 mg of PMS (phenazine methosulfate) (Sigma) in 20 ml of distilled water was added. An example of native gel staining with tetrazolium salt was shown in FIG. 4.

### EXAMPLE 5

### Quantification of Lactate Dehydrogenase-C Expression in Tobacco

A small sample of transgenic or wild type tobacco leaf tissue (˜100 mg) was crushed with a mortar and pestle in the presence of liquid nitrogen. The pulverized tobacco material was transferred to a 1 ml glass tissue grinder (Corning) at 4° C. Immediately following, 500 μl of pre-chilled extraction buffer were added (PBS supplemented with 2 mM PMSF, 2× Complete Protease Inhibitor Cocktail (Roche Biochemicals) and 2 mM EDTA). The tobacco was ground until a homogenous solution was obtained. The solution was transferred to a 2 ml microtube and was centrifuged in an Eppendorf Centrifuge 5417C (Eppendorf) at maximum speed for 20 minutes at 4° C. The supernatant was transferred to a fresh tube and the protein concentration was determined by the Warburg-Christian method. All samples to be tested including the wild type sample were normalized to a common protein concentration by dilution with phosphate buffered saline (PBS) such that accurate measurement of lactate dehydrogenase-C (LDH-C) could be performed.

A sandwich ELISA was developed to quantify the amount of lactate dehydrogenase-C that expresses in the transgenic tobacco plants. Purified polyclonal antibody against lactate dehydrogenase-C provided by Dr. Erwin Goldberg (Northwestern University) was used as the capture antibody. One hundred ng of the antibody were added to a microwell in a 96-well format in a total of 50 μl of capture buffer (100 mM carbonate buffer, pH 9.5). The ELISA plate was incubated at 4° C., washed 3 times with wash buffer (PBST) and extracts from transgenic or wild type plants were added in different concentrations. The plates were incubated for exactly one hour at room temperature and subsequently washed as describe above. Fifty μl of a 1:10,000 dilution of 1 mg/ml biotinyalated rabbit anti-mouse lactate dehydrogenase-C were added and the plates were incubated for one hour. The plates were washed as above and were then incubated for 30 minutes with 50 μl of horseradish peroxidase-neutravidin (Pierce) (1 mg/ml) diluted 1:400 with blocking buffer. The plates were submitted to a last wash and then 100 μl of the substrate ABTS (Moss Inc., Pasadena, Md.) was added. The plates were kept in the dark for 20 minutes and the developing reaction was stopped with 0.5 M oxalic acid (Sigma). The plates were read in an ELISA spectrophotometer at 414 nm.

### EXAMPLE 6

### Preparation of Tobacco Protein Extract for Vaccine Administration

For vaccine trials, tobacco extracts were prepared and partially purified. Possible toxic alkaloids contained in tobacco were detoxified and the lactate dehydrogenase-C in the extract was concentrated so that the administered vaccine volume was not too great to cause animal discomfort.

One hundred grams of leaves were collected from wild type plants or plants expressing greater levels of lactate dehydrogenase-C. The leaves were crushed to a fine powder with a mortar and pestle in the presence of liquid nitrogen. The pulverized tobacco was transferred to a blender and mixed with 250 ml of ice-cold extraction buffer consisted of 2×PBS supplemented with 2 tablets of Complete Protease Inhibitors (Roche Biochemicals), 2 mM PMSF, 2 mM EDTA, 2 mM DTT (Sigma), and 2 mM β-2ME (Sigma). The powder was blended to homogeneity at 4° C. The homogenate was filtered using cheese cloth and was then collected in 50 ml centrifuge tubes. The tubes were centrifuged for 30 minutes at 40,000 g. The supernatant was collected and its volume was measured. It was brought to 40% saturation with enough crushed solid ammonium sulfate (AS) at 4° C. with constant stirring. After an hour of precipitation, the suspension was transferred to centrifuge tubes and was centrifuged for 20 minutes at 40,000 g. The pellet was discarded and the supernatant was brought from 40 to 80% saturation with crushed ammonium sulfate. Protein precipitation was performed overnight with constant stirring at 4° C. The centrifugation process was carried out as described above on the next day. The pellet was resuspended in a minimal volume (˜10 ml) of PBS. The solution was transferred to a SnakeSkin™ 10,000 MWCO dialysis tubing (Pierce Chemical Co. Rockford, Ill.) and was dialyzed overnight in PBS with the buffer changed several times.

The next morning, the dialyzed 40-80 AS fraction was placed in a pre-set ultrafiltration apparatus containing a Diaflo membrane XM300 (Amicon, Beverly, Mass.). The sample was concentrated to about 5 ml. The extract was adjusted to 5% sodium bicarbonate and a few mg of sucrose was added to sweeten the plant extract thereby making it more palatable. The protein concentration was quantified and normalized to the wild type concentration such that equal amounts of protein would be given in extracts from transgenic plants or controls. The samples were aliquoted into small test tubes and stored at −80° C. until further use. The amount of lactate dehydrogenase-C contained in each extract was quantified using the sandwich ELISA developed as described in the previous section.

The extracts prepared in the previous section were thawed and were prepared for vaccine administration by the addition of 10 μg of the mucosal adjuvant cholera toxin (Sigma). A group of 10 mice were given 150 μg of lactate dehydrogenase-C contained in tobacco extract and a group of 5 mice were given half that dose or 75 μg. Groups of 5 mice were given same volume as the 150 μg lactate dehydrogenase-C dose but using extracts from wild type tobacco as negative controls. In addition, to a group of 5 mice 10 μg of cholera toxin was given by itself. The vaccine formulations were administered on days 1, 7, 14, 21, 30, 37, and 68.

To administer the vaccine the sample was loaded into a 1 ml syringe with a stainless-steal animal feeding blunt end needle (Popper & Sons, Inc., New Hyde Park, N.Y.) and the liquid was slowly dispensed into the back of the animal's throat. For two hours prior and 30 minutes after each vaccine administration the animals were deprived of water or food.

### EXAMPLE 7

### Immune Response to Oral Immunization of Tobacco Extracts

To measure antibody responses in serum and vaginal secretions, the animals were bled and vaginally washed on days 0, 25, 32, 55, and 61. To prepare the serum, the mice were first restrained, their tail cut at the tip, and a few (7-10) drops of blood were collected in a tube for each animal. The blood was incubated for 20 minutes at 37° C. and then at 4° C. overnight to complete the clotting process. The blood was spun down in a microcentrifuge at 10000 rpm for 10 minutes. The serum was carefully removed, transferred into a fresh tube, and was either immediately used or kept frozen at −80° C.

Vaginal washes were performed by inserting a blunt pipette tip containing 50 μl of phosphate buffer saltine into the vagina and moving the liquid up and down 10 times. The wash was transferred into a microtube and was kept immediately at 4° C. and stored under the same conditions overnight to allow the large particles sediment to the bottom of the tube.

Production of specific antibodies against lactate dehydrogenase-C in serum and vaginal washes was assessed by indirect ELISA. Briefly, 50 μl of 0.01 mg/ml recombinant lactate dehydrogenase-C in capture buffer (100 mM sodium bicarbonate, pH 9.2) was coated onto 96-wells microtiter plate (Flow Laboratories, McLean Va.) and were further incubated overnight at 4° C. The plates were washed 4 times with PBST (phosphate buffer saline, pH 7.4, 0.5% Tween 20 (Sigma)), incubated at room temperature in blocking buffer (PBST with 1% protease-free BSA (Roche Biochemicals)) for 30 minutes and washed again once. Serum or vaginal washes were added to the wells up to 50 μl in blocking buffer and the plates were incubated for 1 hour at room temperature. When IgA was tested both serum and vaginal washes were diluted 1:10. When IgG was tested the serum was diluted 1:500 and vaginal washes were diluted 1:10. The plates were washed 3 times as described above and then a secondary affinity purified goat anti-mouse IgG or goat anti-mouse IgA coupled to a horseradish peroxidase was added. Both antibodies were obtained from Kirkegaard & Perry Laboratories (Gaithersburg, Md.). For IgG assessment, the antibodies were used at 1:10,000 dilution in blocking buffer. For IgA, a 1:1000 dilution was used. After one hour incubation at room temperature the plates were washed 4 times and 100 μl of hydrogen peroxide-2,2′-Azino-bis-(3-ethylbenzthiazoline-6-sulfonic acid) (ABTS) from Moss Inc. (Pasadena, Md.) was added and incubated for 30 minutes in the dark. The reaction was quenched with 100 μl of 0.5 M oxalic acid. The plates were immediately read at 414 nm in an EL340 Automated Microplate Reader (Bio-Tek Instruments Inc., Winooski VM).

The following references were cited herein:

Alexander and Bialy, Reprod. Fertil. Devel. 6(3):273-280 (1994)

Arakawa et al., Nature. Biotechnol. 16:292-7 (1998).

Babson and Babson, Clin Chem. 19:766-769 (1973).

Bechtold and Pelletier, Methods Mol Biol 82:259-66 (1998).

Corradin et al., J. Immunol. 119:1048-55 (1997).

Fujihashi et al., Vaccine. 20:2431-8 (2002).

Goldberg et al., Fertil. Steril. 35(2):214-7 (1981).

Haq et al., Science 268:714-6 (1995).

Hogrefe et al., J Biol Chem. 262:13155-13162.

Hoiseth and Stocker, Nature. 291: 238-9 (1981).

Horsch et al., Science. 227:1229-31 (1985).

Koncz and Schell, Mol Gen Genet. 204:383-96 (1986).

LeVan and Goldberg, Biochem J. 273:587-92 (1991).

Manson et al., Proc. Natl. Acad. Sci. USA. 93:53335-40 (1996)

O'Hern et al., Biol. Rep. 52:331-9 (1995).

O'Hern et al., Vaccine 16:1761-6 (1997).

Pizza et al., Vaccine 21(17-19):2534-41 (2001)

Rosenwasser and Rosenthal, J. Immunology. 120:1991-8 (1978).

Seamark, Reprod Fertil Dev 13(7-8):705-11 (2001)

Schardl et al., Science 345: 2342-6 (1987).

Schardl et al., Gene 61:1-11 (1987b).

Shelton and Goldberg, J. Reprod. Immunol. 8(4):321-327 (1985)

Van Regenmortel and Daney de Marcillac, Immunol Lett. 17(2):95-107 (1998).

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A genetically modified plant that expresses an immunocontraceptive comprising an egg-specific polypeptide or sperm-specific polypeptide.

2. The genetically modified plant of claim 1, wherein said sperm-specific polypeptide is lactate dehydrogenase-C or an antigenic fragment thereof.

3. The genetically modified plant of claim 2, wherein said lactate dehydrogenase-C is a rodent lactate dehydrogenase-C.

4. The genetically modified plant of claim 3, wherein said rodent lactate dehydrogenase-C is a rat or mouse lactate dehydrogenase-C.

5. The genetically modified plant of claim 2, wherein said antigenic fragment is selected from the group consisting of amino acids 5-17, 44-58, 61-77, 97-110, 180-210, 211-220, 231-243, 283-306, 307-316, and 101-115 of murine lactate dehydrogenase-C.

6. The genetically modified plant of claim 1, wherein said plant is selected from the group consisting of potato, tobacco, corn, banana, wheat, rice, fruit, vegetable, legume, and grain crop.

7. A method of decreasing the fertility of an animal, comprising the step of:
providing said animal the genetically modified plant of claim 1 thus allowing said animal to ingest the plant.

8. The method of claim 7, wherein said animal is selected from the group consisting of mouse, rat, deer, elephants, water buffalo, feral horses, foxes, urban or wild dogs, urban or wild cats, rabbits, and other potentially overpopulated species causing economic damage to society.

9. A genetically modified plant that expresses a sperm-specific polypeptide lactate dehydrogenase-C or an antigenic fragment thereof.

10. The genetically modified plant of claim 9, wherein said lactate dehydrogenase-C is a rodent lactate dehydrogenase-C.

11. The genetically modified plant of claim 10, wherein said rodent lactate dehydrogenase-C is a rat or mouse lactate dehydrogenase-C.

12. The genetically modified plant of claim 9, wherein said antigenic fragment is selected from the group consisting of amino acids 5-17, 44-58, 61-77, 97-110, 180-210, 211-220, 231-243, 283-306, 307-316, and 101-115 of murine lactate dehydrogenase-C.

13. The genetically modified plant of claim 9, wherein said plant is selected from the group consisting of potato, tobacco, corn, banana, wheat, rice, fruit, vegetable, legume, and grain crop.

14. A method of decreasing the fertility of an animal, comprising the step of:
allowing said animal to ingest the genetically modified plant of claim 9.

15. The method of claim 14, wherein said animal is selected from the group consisting of mouse, rat, deer, elephants, water buffalo, feral horses, foxes, urban or wild dogs, urban or wild cats, rabbits, and other potentially overpopulated species causing economic damage to society.
